(19) United States
(12) Patent Application Publication
(10) Pub. No.: US 2003/0008831 A1
(43) Pub. Date: Jan. 9, 2003

(54) Type III collagen compositions

(76) Inventors: Chunlin Yang, Foster City, CA (US); James W. Polarek, Sausalito, CA (US); Thomas B. Neff, Atherton, CA (US)

Correspondence Address: Intellectual Property Department, FibroGen, Inc., 225 Gateway Blvd., South San Francisco, CA, 94080, US

(21) Appl. No.: 10/211,127

(22) Filed: Aug. 2, 2002

Related U.S. Application Data
(63) Continuation of application No. 09/371,401, filed on Aug. 10, 1999.
(60) Provisional application No. 60/095,977, filed on Aug. 10, 1998.

(51) Int. Cl. A61K 38/39

(52) U.S. Cl. 514/21

(57) ABSTRACT

Polymerized type I collagen based compositions for medical use as vascular sealants and wound dressings, the preparation thereof, are described. Prior to polymerization, the collagen monomers are prepared recombinantly whereby chemical modifications of the collagen are not needed to form such monomers. The type I collagen and/or type III collagen compositions, which include gelatin comprised of type I collagen and type III collagen, are useful as medical adhesives for bonding soft tissues and as sealants for a variety of medical uses. In a further aspect of the present invention, the polymerized type I collagen and/or type III collagen compositions include agents which induce wound healing or provide for additional beneficial characteristics desired in a tissue sealant or wound dressing. The compositions of the present invention are also useful in non-adhesive form.

This application is a continuation of U.S. patent application Ser. No. 09/371,401, filed Aug. 10, 1999, which claims the benefit of U.S. Provisional Application No. 60/095,977, filed Aug. 10, 1998.

### FIELD OF THE INVENTION

The present invention is directed to polymerized recombinant type I collagen and/or type III collagen compositions and combinations thereof, including gelatin-based compositions, for medical use as sealants and wound dressings. The present invention is further directed to the preparation of such compositions. These compositions are useful as sealants in a variety of medical applications, including vascular plug type devices, wound closure devices, tendon wraps for preventing the formation of adhesion following surgical procedures, and dressings for use to treat incisions, seeping wounds, and the like, and as medical adhesives for bonding tissues. In a further aspect of the present invention, the compositions include agents which induce wound healing or provide additional beneficial characteristics desired in a tissue sealant. More particularly, the compositions of the present invention are can be used as vascular sealants.

### BACKGROUND OF THE INVENTION

Mechanical, Chemical, Synthetic, and Autologous Adhesion Techniques

The ability to bond biological tissues is an important area of investigation for biomedical researchers. Attempts to provide desired adhesion through purely mechanical bonding have proven to be neither convenient nor permanent. (Buonocore, M., Adhesion in Biological Systems, R. S. Manly, ed., Academic Press, New York, 1970, Chap. 15.) For example, the conventional methods of choice to close incisions in soft tissue following surgery, injury, and the like have been sutures and staples. These techniques and methods are limited by, for example, tissue incompatibility with sutures or staples, which may cause painful and difficult to treat fistulas granulomas and neuromas. Mechanical means can also be limited to being purely adhesive, and thus not fully satisfactory as compared to sealants applied to close wounds that are bleeding or seeping, etc. Sutures and staples may also tend to cut through weak parenchymatous or poorly vascularized tissue. Sutures can leave behind a tract which can allow for leakage of fluids and organisms. Sutures can be further problematic in that the needle for any suture is larger than the thread attached to it and the needle tract is thus larger than can be filled by the thread used to form the sutures.

In addition, limits are imposed by the manual and visual dexterity required on the part of the surgeon and the excessive amount of time needed for the use of sutures or staples in microsurgeries. Furthermore, even when properly applied, staples or sutures are inherently weak or may structurally weaken over time and leak, due to the joints in the gaps between the staples or sutures.

Several investigators have worked on laser closure of wounds. (See, e.g., Abergel, R. P. et al. (1986) J. Am. Acad. Dermatol. 14(5):810-814; Cespanyi, E. et al. (1987) J. Surg. Res. 42(2):147-152; Oz, M. C. et al. (1989) Lasers Surg. Med. 9(3):248-253; Oz, M. C. et al. (1991) Am. Surg. 57(5):275-279; and Oz, M. C. et al. (1993) J. Clin. Laser Med. Surg. 11(3):123-126.) Early efforts concentrated on welding tissues using lasers of different wavelengths applied directly to wound edges and on investigating the microstructural basis of the tissue fusion thus produced. Researchers proposed that a homogenizing change in collagen occurred with interdigitation of altered individual fibrils. (See, e.g., Schober, R. et al. (1986) Science 232(4756):1421-1422.) Investigators explored the idea of heating the collagen fibrils above a threshold level would allow for cross-linking to occur. However, the heat necessary to allow this reaction causes collateral thermal damage. This is undesirable, as even a slight distortion in ocular tissue, for example, may have functional consequences. Also, in the event of laser weld failure, the edges of the tissues may be damaged by the original treatment and cannot be re-exposed to laser energy.

Further research attempted to enhance heat-activated cross-linking by placing a dye in the wound. It was reported that matching the absorbance of the dye with the laser wavelength achieved an adhesive effect with less laser power output and collateral thermal injury. (See, e.g., Chuck, R. S. et al. (1989) Lasers Surg. Med. 9(5):471-477 and Oz, M. C. et al. (1990) J. Vasc. Surg. 11(5):718-725.) Coupling the dye with a protein to create a tissue “solder” was also investigated. The protein commonly used to create a tissue “solder” is fibrinogen, and, in particular, autologous fibrinogen, which is used to avoid problems of the transfer of viral diseases through the use of blood components pooled from different donors. In previous applications, fibrinogen has been obtained as a fraction of whole blood, and thus contains other blood elements, such as clotting factors. Application of such protein-dye mixtures in various animal models proved to be an improvement to the use of dye alone. (See, e.g., Moazami, N. et al. (1990) Arch. Surg. 125(11):1452-1454; and Oz et al. (1990).) However, direct application in humans was prevented due to the need to isolate the necessary protein fibrinogen from the patient prior to the procedure to avoid risks of infection associated with fibrinogen obtained from donor plasma. Other proteins, for example, albumin, were unsatisfactory substitutes, as welds of comparable strength were not achieved.

Comparisons of protein-dye applications and sutured closures show that the protein-dye applications produce less of an inflammatory response and result in greater collagen production, greater mean peak stress at rupture, and better cosmesis. (See, e.g., Wider, T. M. et al. (1991) Plast. Reconstr. Surg. 88(6):1018-1025.) Ophthalmologic applications of such a tissue solder have included the sealing of conjunctival blebs, sclerostomy, closure of retinectomies, and thermokeratoplasty. (See, e.g., Fink, A. J. et al. (1986) Am. J. Ophthalmol. 101(6):695-699 and Latina, M. A. et al. (1990) Arch. Ohthalmol. 108(12):1745-1750.)

Due to the deficiencies and limitations of mechanical means, such as the above mentioned sutures, staples, and laser techniques, efforts were made to develop synthetic polymers, such as, for example, cyanoacrylates, as biomedical adhesives and sealants. These plastic materials, however, induce inflammatory tissue reactions. In addition, the ability of these materials to establish permanent bonding under physiological conditions has yet to be fully realized.

The known toxicity associated with synthetic adhesives has led to the development of biologically-derived adhesives as bonding materials. Fibrin-based glues, for example, have commanded considerable attention. (See, e.g., Epstein, G. H. et al. Ann. Otol. Rhinol. Laryngol. 95:40-45 (1986); Kram, H. B et al. Arch. Surg. 119:1309-1311 (1984); Scheele, J. et al. Surgery 95:6-12 (January 1984); and Siedentop, K. H. et al. Laryngoscooe 93:1310-1313 (1983) for general discussion of fibrin adhesives.) Commercial fibrin tissue adhesives are derived from human plasma and thus pose potential health risks, such as adverse immunogenic reactions and transmission of infectious agents, such as, for example, Hepatitis B virus. Moreover, the bond strength imparted by such adhesives is relatively weak compared to collagen adhesives. (See, for example, De Toledo, A. R. et al. Assoc. for Res. in Vision and Ophthalmology, Annual Meeting Abstract, Vol. 31, 317 (1990).) Accordingly, there is a need for safe and effective biologically compatible tissue adhesives for biomedical applications.

More recently, combination products have been devised for use as tissue adhesives and sealants. The use of a combination of three separately prepared substances, human fibrinogen cryoprecipitate, thrombin in the presence of calcium ion, and Factor XIII concentrate, to obtain a glue for use in skin graft applications, myringoplasty, repair of dural defects, hemeostatis after tonsillectomy, and tracheoplasty, has been described. (See, Staindl (Ann. Otol (1979) 88:413-418).) In this same time frame, Immuno-AG (Vienna, Austria) began producing and commercializing a two-component “fibrin seal” system, wherein one component contains highly concentrated human fibrinogen, Factor XIII, and other human plasma proteins, all prepared from pooled blood, and the other component supplies thrombin and calcium ions. The two components are added together in the presence of a fibrinolysis inhibitor. After application, coagulation and fibrin cross-linking occur. Eventually, the seal may lyse in the process of healing of the wound or trauma which accompanies the reconstruction of the tissue. The development of an applicator device for this system, which mixes and applies the two components of the system simultaneously, has been described. (Redl, H., et al., “Biomaterials 1980,” Winter, G. D., et al., eds. (1982), John Wiley & Sons, Ltd., at page 669-675.) These combination systems and their uses have been described widely. (See, e.g., Seelich, T., J Head and Neck Pathol (1982) 3:65-69; O'Connor, A. F., et al., Otolaryngol Head Neck Surg (1982) 90:347-348; Marquet, J., J Head and Neck Pathol (1982) 3:71-72; Thorson, G. K., et al., J Surg Oncol (1983) 24:221-223.) It has also been reported that the addition of barium ion to this fibrin glue system in the treatment of a bleeding duodenal sinus facilitates follow-up surveillance. (See, for example, McCarthy, P. M., et al., Mayo Clin Pros (1987) 62:317-319 and Portmann M., J Head and Neck Pathol (1982) 3:96; Panis, R., ibid., 94-95.)

Efforts have recently focused on methods which seek to avoid health issues raised by the use of blood plasma-derived products in commercially available tissue adhesive products and systems. Attempts have been made to isolate an autologous counterpart of the fibrinogen-containing component. (See, for example, Feldman, M. C., et al., Arch Otolaryngol-Head and Neck Surg (1988) 114:182-185; Feldman, M. C., et al., Arch Ophthalmol (1987) 105:963-967; Feldman, M. C., et. al., M J Otolog (1988) 9:302-305; and Silberstein, L. E., et al., Transfusion (1988) 28:319-321.) Use of autologous fibrinogen preparations also has obvious limitations.

Vascular Sealants

One critical aspect of tissue adhesion is the sealing of wounds, and, in particular, vascular punctures and other vascular wounds resulting from, for example, surgery.

For example, percutaneously accessing major vascular structures is a key step in a variety of diagnostic and therapeutic procedures, including Percutaneous Transluminal Coronary Angioplasty (PTCA), Percutaneous Coronary Angiography, and Percutaneous Coronary Atherectomy. In a percutaneous intravascular procedure, access to the vascular space is generally obtained using the Seldinger technique where, first, a hollow needle is used to create a puncture wound through the skin, the underlying muscle tissue, and the wall of a selected blood vessel, such as the femoral artery. Next, a guidewire is inserted through the tubular needle until its distal end is located in the blood vessel, at which time the needle is stripped off of the guidewire and replaced with an introducer sheath and dilator. The introducer sheath typically includes a self-sealing hemostatic valve on its proximal end for sealing around the guidewire. The guidewire is then advanced into the vascular space through the introducer and directed to a preselected area of the vascular system. Once the guidewire is positioned, a catheter is advanced over the guidewire to the desired area.

Once the procedure has been completed and the catheter and the introducer sheath are removed from the puncture site, there may be profuse bleeding, especially when the patient has been on anticoagulant therapy, such as heparin, coumadin, aspirin, or thrombolytic agents. The most common method used to prevent post-procedure bleeding at the access site involves the application of direct pressure to the perforation site until normal physiologic pathways have sealed the access site. There are several problems with this method. Firstly, the pressure application technique may fail to prevent hemorrhage. Such a hemorrhage may be life-threatening or can lead to a large hematoma. A large hematoma in the groin, for instance, may compromise the major nerve supply to the anterior lower extremity.

Secondly, the pressure application technique extends the length of the in-hospital stay. For example, a PTCA may be completed in 2 to 3 hours, but the patient will typically be hospitalized for several additional hours or overnight to allow the access site to seal physiologically. During this extended hospital stay the patient is required to stay immobile, often with a sand bag taped to the patient's thigh, such as in the case of femoral artery access.

These and other complications are exacerbated when PTCA procedures are performed in elderly patients who commonly have arteries with reduced natural elasticity. The access perforation in a relatively inelastic artery does not contract or shrink upon itself to the same extent as in an artery of normal elasticity. The resulting undeflected perforation is typically two to three times larger than an access perforation in a normal artery, further complicating the initiation of hemostasis and the normal physiologic sealing of the access site.

More than 500,000 PTCAs were performed worldwide in 1992 (Cowen Report, March 1993), and several times that number of other procedures requiring accessing major vascular structures percutaneously were also performed. Thus, the increased length of in-hospital stay necessitated by the pressure application technique considerably increases the expense of procedures requiring such vascular access.

A technique that would allow faster and safer sealing of a vascular access site would save a significant amount of health care resources. Medical literature has addressed the problem of achieving hemostasis following removal of a percutaneously applied intravascular introducer in such uses as angiography or angioplasty by a number of divergent means. U.S. Pat. No. 5,290,310 describes a device for delivering a collagen plug subcutaneously against a penetration site in a wall of a blood vessel. An instrument containing a toroidal-shaped collagen plug within a barrel thereof is made to surround the exterior of a tubular introducer. The instrument includes a pusher mechanism for ejecting the collagen plug into the puncture wound and against the exterior wall of the blood vessel at the site of the puncture. This device relies upon a collagen plug which is derived from animal sources and is therefore comprised primarily of heterotrimer type I collagen.

U.S. Pat. No. 5,129,882 also discloses a surgical implement for injecting a hemostatic agent in a puncture wound by routing the injection device through the lumen of the introducer sheath after it has been retracted sufficiently so that the distal end thereof is no longer in the blood vessel. Deploying a plunger, the hemostatic agent is forced out of the instrument and against the exterior wall of the artery proximate the puncture wound.

U.S. Pat. Nos. 4,744,364, 4,852,974, 4,890,612, 5,021,059, and 5,222,974 each describe a method and apparatus for effecting hemostasis by inserting an anchoring device through the puncture wound and into the blood vessel while using a filament attached to the anchoring device to inject an appropriate sealant into the wound. The anchoring device prevents entrance of the sealing material into the blood vessel and serves as an anchor and guide for addressing selected vessels.

Still other devices for injecting a hemostatic agent into a puncture wound following a vascular procedure are described in U.S. Pat. Nos. 5,281,197, 4,838,280, 5,192,300, and 4,738,658, and in published European Patent Application 0 476 178A1.

Collagen and Gelatin as Biomaterials

Collagen, the major connective tissue protein in animals, possesses numerous characteristics not seen in synthetic polymers. Characteristics of collagen include good compatibility with living tissue, promotion of cell growth, and absorption and assimilation of implantations. (See, e.g., Shimizu, R. et al. Biomat. Med. Dev. Art. Org., 5(1): 49-66 (1977).) These same characteristics are also true of gelatins, which are derived from collagens.

Various applications of collagen as a biomaterial are being tested, for example, in dialysis membranes of artificial kidney, artificial corneas, vitreous body, artificial skin and blood vessels, hemostatic agents, soft contact lenses, and various surgical applications. (Sterzel, K. H. et al. Ameri. Soc. Artif. Int. Organs 17:293 (1971); Rubin, A. L. et al. Nature 230:120 (1971); Chvapil, M. et al. Int. Rev. Conn. Tiss. Res. 6:1-61 (1973); Dunn, M. et al. Amer. Soc. Artif. Int. Organs 17:421 (1971); Krajicek, M. et al. J. Surg. Res. 4, 290 (1964); and U.S. Pat. Nos. 4,581,030, 4,215,200, 4,264,155, 4,264,493, 4,349,470, 4,388,428, 4,452,925, and 4,650,616.)

Natural collagen fibers, however, are basically insoluble in mature tissues because of covalent intermolecular cross-links that convert collagen into an infinite cross-linked network. Dispersal and solubilization of native collagen can be achieved by treatment with various proteolytic enzymes which disrupt the intermolecular bonds and remove immunogenic non-helical end regions without affecting the basic rigid triple-helical structure which imparts the desired characteristics of collagen. (See, e.g., U.S. Pat. Nos. 3,934,852, 3,121,049, 3,131,130, 3,314,861, 3,530,037, 3,949,073, 4,233,360, and 4,488,911 for general methods for preparing purified soluble collagen.)

Various methods and materials have been proposed for modifying collagen to render it more suitable as biomedical adhesives. (See, e.g., De Toledo, A. R. et al. Assoc. for Res. in Vision and Ophthalmology, Annual Meeting Abstract, Vol. 31, 317 (1990); Lloyd et al. “Covalent Bonding of Collagen and Acrylic Polymers,” American Chemical Society Symposium on Biomedical and Dental Applications of Polymers, Polymer Science and Technology, Vol. 14, Plenum Press (Gebelein and Koblitz eds.), New York, 1980, pp. 59-84; Shimizu et al., Biomat. Med. Dev. Art. Org., 5(1): 49-66 (1977); and Shimizu et al., Biomat. Med. Dev. Art. Org., 6(4): 375-391 (1978), for general discussion on collagen and synthetic polymers.) In many instances, the prior modified collagen-based adhesives suffer from various deficiencies, including (1) cross-linking/polymerization reactions that generate exothermic heat, (2) long reaction times, and (3) reactions that are inoperative in the presence of oxygen and physiological pH ranges. (See, e.g., Lee M. L. et al. Adhesion in Biological Systems, R. S. Manly, ed., Academic Press, New York, 1970, Chap. 17.) Moreover, many of the prior modified collagen-based adhesives contain toxic materials, rendering them unsuitable for biomedical use. (See, for example, Buonocore, M. G. (1970) and U.S. Pat. No. 3,453,222.)

Additionally, the use of collagen-based adhesives also presents immunological concerns as such adhesives have been derived from animal sources and typically bovine sources. Studies with respect to the use of such collagens as injectible devices have reported minor inflammatory responses. More recently, potential issues regarding the transmission of disorders to humans related to bovine spongiform encephalopathy (i.e., mad cow disease) have focused attention, especially in Europe, to limiting the use of animal, and particularly, bovine-sourced materials.

Notwithstanding these deficiencies, certain collagen-based adhesives, reportedly having appropriate adhesive strength and utility in many medical applications, particularly involving soft tissues, have been described. (See, e.g., U.S. Pat. Nos. 5,219,895, 5,614,587, 5,582,834, 5,575,997, 5,354,336, and 4,600,574.) The literature identifies the use of type I and type Ill collagens in collagen-based adhesives wherein purified type I and type III collagens are chemically modified to form monomers soluble at physiological conditions and polymerized to form compositions having adhesive and sealant properties. Notably, the reports are limited to collagen-based adhesives composed of collagens derived from natural sources which represent a collagen mixture. For example, type I collagen as isolated from natural sources typically contains approximately 10-20% type III collagen, as well as other collagens, depending upon the tissue source used, and about 90-80% type I collagen. With respect to the “type I collagen” mixtures, the literature further teaches only the use of collagen as an adhesive as a consequence of its structural characteristics, or, alternatively, the use of predominantly type I collagen heterotrimers, as compared to type I collagen homotrimers, which have been implicated in the epithelial cell attachment. (See, e.g., Ghersi, et al. 1989, Eur. J. Cell Biol. 50:279-84).

Available reports do not refer to type III collagen, the unexpected hemostatic characteristics of type III collagen, or the use of recombinant collagens which would allow the first chemical modification step, as described in the art, to be avoided.

In summary, there is a need in the art for compositions useful as sealants and wound dressings that permit faster and safer healing, that minimize the risks of infection from donor, including non-human, sources, that increase convenience and permanence, that minimize demands on surgical resources and time, and that demonstrate superior biocompatibility. In addition, there is a need for biologically-derived adhesives that offer improved convenience and permanence over currently available formulations, and that promote less-invasive treatment, resulting in improved patient comfort and shorter time under medical supervision. Additionally, such compositions would preferably offer improved bond strength. There is also a need for non-adhesive compositions that provide the above-named advantages of safer and more effective healing and that are biologically compatible.

### SUMMARY OF THE INVENTION

The present invention includes biologically compatible type III collagen and/or type I collagen products with sealant properties which can be formed using soluble recombinantly derived type III collagen and/or type I collagen monomers or gelatin derived from type III collagen and/or type I collagen monomers (the collagen and gelatin products are collectively hereinafter referred to as “collagen”), wherein said monomers are polymerized to form a type III collagen and/or type I collagen composition having sealant properties. Preferably, the collagen is human and is derived using recombinant technology. Type III collagen was selected for its unexpectedly superior hemostatic characteristics, as compared to other collagen types. Type I collagen was selected for its structural characteristics, as well as for the hemostatic properties of certain type I collagen forms (e.g., type I collagen homotrimers). The polymerization reaction may be initiated with an appropriate polymerization initiator such as a chemical oxidant, ultraviolet irradiation, a suitable oxidative enzyme, or atmospheric oxygen. Additionally, cross-linking agents including glutaraldehyde, dye-mediated photooxidation, PEG and its derivatives, acyl azide, plyepoxy fixatives, oxidized starch (periodate) and water-soluble carbodiimide may be used in the polymerization process to form a collagen composition having sealant properties.

For purposes of optimizing the sealant and adhesive properties of the recombinant collagen product by optimizing the structural stability and the hemostatic characteristics of the product, the product is comprised of a combination of pure recombinant type I and type III collagen The ratio of pure recombinant type III collagen to pure recombinant type I collagen (heterotrimer) is preferably about 30% and greater type III collagen to about 70% or less type I collagen (heterotrimer). More preferably, the ratio of pure recombinant type III collagen to pure recombinant type I collagen (heterotrimer) is about 30% to about 50% type III collagen to about 70% to about 50% type I collagen (heterotrimer). Most preferably, the ratio of pure recombinant type III collagen to pure recombinant type I collagen (heterotrimer) is about 30% to about 40% type III collagen to about 70% to about 60% type I collagen (heterotrimer).

With respect to compositions comprised of type I collagen homotrimer, the ratio of pure recombinant type I collagen homotrimer to a combination of recombinant type I collagen heterotrimer and recombinant type III collagen is about 90:10. More preferably, the ratio of pure recombinant type I collagen homotrimer to a combination of recombinant type I collagen heterotrimer and recombinant type III collagen is about 75:25. Most preferably, the ratio of pure recombinant type I collagen homotrimer to a combination of recombinant type I collagen heterotrimer and recombinant type III collagen is about 50:50.

It is the object of this invention to provide for a pure recombinant type III collagen tissue sealant, a pure recombinant type I collagen tissue sealant, or a pure recombinant type I collagen and type III collagen tissue sealant, free from other collagen types, having at least one of the following characteristics and capabilities:

(i) Hemostasis. The sealant acts as a hemostatic barrier and reduces the risk of serum, lymph, and liquid leakage. As type III collagen possesses inherently hemostatic properties, its use in a hemostatic device provides an improvement over known fibrin sealants. Type I collagen also possesses some hemostatic properties.

(ii) Gluing. Due to its adhesive properties, the sealants of the present invention connect tissues by forming a strong joint between them and adapt uneven wound surfaces. The gluing effect is increased by a combination of agents, such as those described below, and type III collagen and/or type I collagen.

(iii) Wound healing. The sealant promotes the growth of fibroblasts which, in combination with efficient hemostasis and adhesion between the wound surfaces provides for an improved healing process. (See also, Ghersi, et al. 1989, Eur. J. Cell Biol. 50:279-284 (comparing characteristics of homotrimer and heterotrimer type I collagen).) The use of the present compositions as anti-adherence/wound healing compositions is expected to result in a normal (regenerative) tissue rather than scar tissue, i.e., optimal wound healing. Furthermore, such compositions also reduce the inflammatory response.

Accordingly, it is an object of the present invention to provide polymerized type III collagen and/or type I collagen compositions as safe, effective biological adhesives with appropriate adhesive strength for biomedical applications, particularly those involving soft tissues. More specifically, the present invention is directed to compositions useful in sealing punctures and incisions in large blood vessels and the heart. The polymerized materials may assume a number of sizes and shapes consistent with their intended biomedical applications, which include use in ophthalmology, plastic surgery, orthopedics, and cardiology. The vascular sealant compositions of the present invention, comprising type III collagen and/or type I collagen, may be used alone or in combination with a tissue sealant device, including, for example, the devices set forth in U.S. Pat. Nos. 5,782,860 (issued Jul. 21, 1998), 5,759,194 (issued Jun. 2, 1998), and 5,728,132 (issued Mar. 17, 1998).

In another object of the invention, the type III collagen and/or type I collagen composition is further comprised of agents which will confer additional desirable characteristics for a vascular sealant or wound dressing. For example, fibrin, fibrinogen, thrombin, calcium ion, and Factor XIII may be included in the composition to better effect the formation of a three-dimensional network of polymerized collagen. In yet another object of the invention, the recombinant type III collagen compositions incorporate compounds having wound healing capabilities. In one embodiment, the compound is connective tissue growth factor and is incorporated in the composition to effect slow-release of the compound to the wound. In a second embodiment of the invention, the drug improves vascularization, for example, tumour necrosis factor, as described in U.S. Pat. No. 4,808,402 (issued Feb. 28, 1989).

### BRIEF DESCRIPTION OF THE DRAWINGS

FIG. 1 shows SDS-PAGE analysis of recombinant type III collagen produced by Pichia pastoris.

FIG. 2 sets forth data relating to the biocompatibility of recombinant type III collagen and a commercially available collagen hemostat.

FIG. 3 sets forth data relating to platelet aggregation experiments of recombinant type III collagen and bovine type I collagen.

FIG. 4 sets forth data relating to the bleeding time of spleen treated with recombinant type III collagen and bovine type I collagen.

FIG. 5 sets forth data showing SDS-PAGE analysis of bovine type I collagen cross-linked with water-soluble carbodiimide.

FIG. 6 sets forth data showing SDS-PAGE analysis of recombinant type III collagen cross-linked with water-soluble carbodiimide.

### DESCRIPTION OF THE INVENTION

It is understood that the present invention is not limited to the particular methodology, protocols, cell lines, vectors, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references cited herein are incorporated by reference herein in their entirety.

Definitions

As employed herein, the term “biologically compatible” refers to recombinant type III collagen and/or type I collagen modified in accordance with the present invention (i.e., a polymerized type III collagen recombinant product) which is incorporated or implanted into or placed adjacent to the biological tissue of a subject and more particularly, does not deteriorate appreciably over time or induce an immune response or deleterious tissue reaction after such incorporation or implantation or placement.

As employed herein, the term “pure recombinant type I collagen” refers to type I collagen manufactured by recombinant techniques which is substantially free from other collagen types. Unless otherwise specifically referenced, the term pure recombinant type I collagen includes both type I collagen homotrimers and type I collagen heterotrimers and mixtures thereof. The term includes any other forms of recombinant type I collagen and any modifications made thereto that may be categorized as a subset of collagen, such as gelatins. The term excludes type I collagen isolated from natural sources.

As employed herein, the term “pure recombinant type III collagen” refers to human type III collagen manufactured by recombinant techniques which is substantially free from other collagen types. The term includes any other forms of recombinant type III collagen and any modifications made thereto that may be categorized as a subset of collagen, such as gelatins. The term excludes type III collagen isolated from natural sources.

As employed herein, the term “substantially free” refers to a recombinant collagen type that is substantially pure of any other collagen type or unmixed with any other collagen type, and is preferably at least 90% free from other collagen types.

As employed herein, the term “vascular sealant” refers to any composition useful in closing vascular wounds, including plugs, which possesses hemostatic properties.

As employed herein, the term “wound” refers to any opening in the skin, mucosa or epithelial linings, most such openings generally being associated with exposed, raw or abraded tissue. There are no limitations as to the type of wound or other traumata that can be treated in accordance with this invention, such wounds include, but are not limited to, first, second, and third degree bums (especially second and third degree); surgical incisions, including those of cosmetic surgery; wounds, including lacerations, incisions, and penetrations; and ulcers, including decubital ulcers (bed-sores) and ulcers or wounds associated with diabetic, dental, haemophilic, malignant, and obese patients. Although the primary concern is the healing of major wounds by neovascularization, it is contemplated that the present invention may also be useful for minor wounds, and for cosmetic regeneration of epithelial cells. Preferably, the wounds to be treated are burns and surgical incisions, whether or not associated with viral infections or tumors

Production of Type I Collagen and Type III Collagen Monomers

Types of collagen useful in forming the biologically compatible collagen products of the invention with adhesive and hemostatic properties are recombinant type I collagen and recombinant type III collagen. Monomeric soluble type I collagen and type III collagen are obtained by recombinant processes, including processes involving the production of type III collagen in transgenic animals. Such recombinant processes are set forth, for example, in U.S. Pat. No. 5,593,859, which is incorporated herein by reference. Preferably, type I collagen or type III collagen will be recombinantly manufactured by culturing a cell which has been transfected with at least one gene encoding the polypeptide comprising type I collagen or type III collagen and genes encoding the α and β subunits of the post-translational enzyme prolyl 4-hydroxylase and purifying the resultant collagen monomer therefrom. Preferably, the monomeric soluble type I collagen and type III collagen material exhibits a viscous consistency and varying degrees of transparency and clarity.

Polymerization of Type I Collagen and Type III Collagen Monomers

The recombinant type I collagen and recombinant type III collagen solution may be subsequently subjected to polymerization or cross-linking conditions to produce the polymerized collagen composition of the present invention. Polymerization may be carried out using irradiation, e.g., UV, gamma, or fluorescent light. UV irradiation may be accomplished in the short wave length range using a standard 254 nm source or using UV laser sources. With a standard 254 nm source, 4 to 12 watts, polymerization occurs from 10 to 40 minutes, preferably 20 to 30 minutes, at an exposure distance of from 2.5 to 10 cm, preferably from 2.5 to 5 cm distance. Excess UV exposure will begin to depolymerize the collagen polymers. Polymerization using gamma irradiation can be done using from 0.5 to 2.5 Mrads. Excess gamma exposure will also depolymerize collage polymers. Polymerization in the presence of oxygen can be achieved by adding an initiator to the fluid prior to exposure. Non-limiting examples of initiators include sodium persulfate, sodium thiosulfate, ferrous chloride tetrahydrate, sodium bisulfite, and oxidative enzymes such as peroxidase or catechol oxidase. When initiators are employed, polymerization occurs in 30 seconds to 5 minutes, usually from 1 to 3 minutes.

The polymerizing agent is preferably UV irradiation. However, the polymerization or cross-linking of the monomeric substituents can be carried out by any of the methods well known in the art, including simply exposing the material to atmospheric oxygen, although the rate of polymerization is appreciably slower than in the case of UV irradiation or chemical agents.

Other agents may also be useful in the polymerization process. For example, to improve the cohesive strength of adhesives formed from the compositions of this invention, difunctional monomeric cross-linking agents may be added to the monomer compositions of this invention to effect polymerization. Such cross-linking agents are known in the art, for example, in U.S. Pat. No. 3,940,362, which is hereby incorporated by reference herein.

Additionally, polymerization methods and cross-linking agents such as glutaraldehyde, dye-mediated photooxidation, PEG and its derivatives, acyl azide, plyepoxy fixatives, oxidized starch (periodate), and water-soluble carbodiimide, all well known in the art, may be used to produce the polymerized collagen composition of the present invention. (See, e.g., U.S. Pat. Nos. 4,615,794, 5,444,154, 4,500,453, 5,702,818, 5,415,938, 5,308,641, 5,264,551, 5,258,501, 5,258,481, 4,427,808, and 4,272,610.)

Moreover, the use of polyaldehyde compositions to effectuate polymerization can be also utilized. (See, for example, PCT WO 97/29715 and EP 747,066 A2.)

Formation of Gelatin

The recombinant collagen protein of the present invention may be further modified and processed into gelatin using procedures known in the art. (See, e.g., Veis, 1965, International Review of Connective Tissue Research, “The Physical Chemistry of Gelatin”, Academic Press, New York and London.) For example, a common feature of all standard collagen to gelatin conversion processes is the loss of the secondary structure of the collagen protein, and in the majority of instances, an alteration in either the primary or tertiary structure of the collagen. The collagens of the present invention can be processed using different procedures depending on the type of gelatin desired.

In one approach, modifications may occur to unpurified collagen or procollagen present in the cell mass or in the culture medium or any further modifications can be made to the purified collagen as described above. For example, recombinant collagen or procollagen may be modified and processed into recombinant gelatin. Gelatin may be produced directly from the cell mass or the culture medium by taking advantage of gelatin's solubility at elevated temperatures and stability at conditions of low or high pH, low or high salt concentration, and high temperatures. For example, the cell mass or culture medium may further be treated to extract gelatin by denaturing the triple helical structure of collagen using detergents, heat or denaturing agents. (See, e.g., Vies, supra, inter alia.) Operations well established for the manufacture of tissue-derived gelatin can be applied to the production of recombinant gelatin. This includes, but is not limited to, treatments with strong alkali or strong acids, heat extraction in aqueous solution, ion exchange chromatography, cross-flow filtration, and heat drying.

Type I Collagen and Type III Collagen Compositions

The compositions of the present invention are comprised of polymerized type I collagen and type III collagen wherein said compositions are manufactured by a process comprising the steps of: (1) production of type I collagen and type III collagen monomers by the recombinant methods described above; and (2) polymerization of such monomers. In addition, where the final composition is a gelatin-based sealant or wound dressing, the process includes a step wherein the collagen is converted into gelatin.

For purposes of optimizing the sealant and adhesive properties of the recombinant collagen product by optimizing the structural stability of the product as well as the hemostatic characteristics of the product, the product is comprised preferably of a combination of pure recombinant type I and type III collagen. The ratio of pure recombinant type III collagen to pure recombinant type I collagen (heterotrimer) is about 30% and greater type III collagen to about 70% or less type I collagen (heterotrimer). More preferably, the ratio of pure recombinant type III collagen to pure recombinant type I collagen (heterotrimer) is about 30% to about 50% type m collagen to about 70% to about 50% type I collagen (heterotrimer). Most preferably, the ratio of pure recombinant type III collagen to pure recombinant type I collagen (heterotrimer) is about 30% to about 40% type III collagen to about 70% to about 60% type I collagen (heterotrimer).

The appropriate ranges of concentrations of components in the tissue sealants and adhesives of the present invention can be determined by methods well known in the art. (See, e.g., Haraski, H. et al. (1999) Volume XXII, Society for Biomaterials, pages 158 through 159; Fasman, G. D., ed. (1989) Practical Handbook of Biochemistry and Molecular Biology, Section 1, pages 126 through 130; U.S. Pat. No. 5,834,232 (issued Nov. 10, 1998); Sierra, D. H. et al. (1992) J. Appl. Biomater. 3(2):147-151; Martinowitz, U. and R. Saltz (1996) Curr. Opin. Hematol. 3(5):395-402; and Siriex, D. (1998) Ann. Vase. Surg. 12(4):311-316.) The actual proportions of the collagen components of the compositions of the present invention will depend on the addition of other agents to the compositions and on the desired use of the compositions. The determination of suitable proportions for particular compositions is within the level of skill in the art, and this invention contemplates the various combinations that can be reached.

The compositions of the present invention may be further comprised of other agents useful in gluing or sealing vascular tissues, and more generally, soft tissue. For example, in addition to recombinant type I collagen and/or recombinant type III collagen protein, the composition will preferably comprise transglutaminases such as Factor XIII and/or fibrin/fibrinogen/fibronectin and/or plasminogen. The suitable concentrations of these components can be selected by methods-well known in the art. For example, fibrinogen can be present in plasma concentrations, such as from about 1.5 to about 4.0 mg/ml, or higher. Fibrinogen can also be present in lower concentrations, for example, to monitor performance.

Preferably, the composition will also include clotting enzymes, i.e. thrombin, especially in combination with bivalent calcium, such as calcium chloride. The concentration of calcium chloride can vary, for example, from between 40 to 200 mM, depending on the specific purpose of the tissue adhesive composition. High concentrations of calcium chloride inhibit fibroblast growth and are therefore preferred for anti-adherence applications (fibronectin, which stimulates the growth of fibroblasts, can be absent in such compositions). It may further be valuable to include a fibrinolysis inhibitor, such as a plasmin inhibitor, e.g. aprotinin, aprilotinin, alpha-2-antiplasmin, alpha-2-macroglobulin, alpha-1-antitrypsin, epsilon-aminocaproic or tranexamic acid, or a plasmin activator inhibitor, e.g., PAI-1 or PAI-2.

While the proportions of the previously known ingredients in the tissue adhesive compositions of the invention may be selected according to methods well-known in the art, the necessary amount of the viscosity-enhancing polymer can readily be determined by a person skilled in the art depending on the particular polymer and the intended use form. Thus, if the concentration and/or molecular weight of the viscosity-enhancing polymer is too low, the viscosity increase will be insufficient, and a too high concentration and/or molecular weight will inhibit the fibrin polymerization and the adhesion to the tissue.

By increasing the thrombin concentration, the polymerization of composition of the present invention may be quickened, reducing the time until the glue sets. At low thrombin concentrations, for example, the fibrin of the composition will remain more or less fluid for several minutes after application. A further beneficial effect of increasing the viscosity with a viscosity-enhancing polymer in accordance with the invention is that lower concentrations of thrombin, required in situations where the parts to be sealed require subsequent adaptation even on non-horizontal surfaces, can be used.

Likewise, the compositions of the present invention may, rather than including a combination of the agents described herein, include a fusion protein wherein the type I collagen and/or type III collagen and, for example, fibrin, are combined to form one molecule. Such fusion proteins may be manufactured according to recombinant techniques described herein.

In a further embodiment of the invention, the composition of the present invention includes agents useful in wound healing, either by inducing or promoting the formation of tissue, or, alternatively, by limiting the formation of fibrotic adhesions. Such agents include antibiotics, or growth factors, such as connective tissue growth factor, described in, for example, U.S. Pat. Nos. 5,408,040 and 5,585,270, incorporated herein by reference. In another embodiment of the invention, the drug improves vascularization, for example, tumour necrosis factor, as described in U.S. Pat. No. 4,808,402 (issued Feb. 28, 1989).

With respect particularly to the vascular sealant aspect of the present invention, vascular sealant compositions comprising collagen type III and/or I may be used alone or in combination with a tissue sealant device, including, for example, the devices set forth in U.S. Pat. Nos. 5,782,860 (issued Jul. 21, 1998), 5,759,194 (issued Jun. 2, 1998), and 5,728,132 (issued Mar. 17, 1998).

Fields Of Use

The polymerized type III collagen and/or type I collagen products of the present invention may be useful to produce mechanical sealants and adhesive systems.

Vascular Adhesive Systems

Fields of application include, but are not limited to, general surgery, dentistry, neurosurgery, plastic surgery, thorax and vascular surgery, abdominal surgery, orthopaedics, accident surgery, gynaecology, urology, and opthalmology. The collagen sealants of the present invention have also been used for local application of drugs, such as antibiotics, growth factors, and cytostatics.

Sealant Films and Wound Dressings

In one aspect of the invention, the polymerized collagen products can be made in the form of a sealant film. A collagen-based film will be flexible and elastic with the consistency and feel of plastic film, but can exhibit high biological compatibility. Uses of sealant films include, but are not limited to, prevention of adhesion formation following tendon surgery (i.e., use as a wrap around tendons), use as a synthetic tympanic membrane, and uses as substitute facial tissue and wound dressing components. Additional examples of potential uses of sealant films include, treatment of corneal abrasions, wound closure, coating of catheters and instruments, and use as a material to prevent adhesion formation in tissues and tendons (e.g., peritoneal cavity).

Further embodiments of the present invention include sealant and adhesive formulations which can be used in systems specific for delivery of numerous drugs and pharmaceutical compositions, including growth factors, antibiotics, and other biologically beneficial compounds. Such materials can be added to the collagen adhesive or sealant to promote cell migration, cell adhesion, and wound healing.

Angioplasty and Angiography

Angiography is a diagnostic procedure whereby dye is injected into an artery, preferably the femoral artery, to detect the presence or absence of coronary disease. Angioplasty, also known as PCTA, is a therapeutic procedure which involves the inflation of a balloon in an artery, such as the coronary artery, for the purpose of relieving arterial blockages. After puncturing the femoral artery, a balloon-catheter is introduced through the femoral artery and navigated through to the coronary artery blocked by atherosclerosis (plaque). Once in position, the balloon is inflated and deflated several times in an effort to open the artery by pushing the fatty material against the vessel walls, allowing for blood to circulate to the affected regions of the heart muscle. Various types of balloon catheters are commonly used in angioplasty and angiography, including over-the-wire catheters which utilize an independent guidewire to the site of the disease; fixed-wire catheters, which combine a balloon catheter with a guidewire into one device; rapid-exchange or single-operator exchange catheters, which are over-the-wire catheters that can be exchanged more conveniently than standard over-the-wire catheters; and perfusion catheters, which allow blood flow during the procedure. A rotational tip catheter removes plaque buildup on arterial walls. These devices utilize a technique called differential cutting. Calcified material is rendered into microscopic particles without damaging the artery due to the elastic nature of the arterial walls.

Angioplasty is a more invasive and complicated procedure than angiography, requiring the insertion of a larger sheath than that used in angiography. The sheath is used as a vehicle for introducing the catheter into the artery. Additionally, angioplasty also requires the use of blood thinners, such as heparin, to prevent clotting during and after the surgical procedure. The anti-clotting agent prevents the body's natural sealing/clotting mechanism and, thus, sealing punctures requires a significant length of time.

According to the present invention, after withdrawing the catheter and other invasive devices from the artery, an adhesive applicator may optionally be inserted into the sheath and placed into a position near to or contacting the puncture in the artery. During the procedure, manual or mechanical pressure is applied to the artery to reduce the flow of blood at the puncture site. If possible, excess blood/fluid is removed from the puncture site. Subsequently, recombinant type III collagen and/or recombinant type I collagen monomer of the present invention may be applied to the puncture on the external surface of the artery and/or within the puncture track. The monomer then is polymerized and/or cross-linked by the techniques described herein, for example, UV irradiation, such that polymerization takes place within 0 to 300 seconds, preferably within 0 to 120 seconds, more preferably within 0 to 30 seconds, and most preferably 3 to 10 seconds. By applying the collagen monomer composition on the outside of the artery, the incidence of embolism (blockage of the artery or circulatory system) is virtually eliminated. Alternatively, polymerization may be achieved according to the methods set forth in PCT WO 97/29715 and EP 747,066 A2, incorporated herein by reference.

Alternatively, a polymerized type III collagen and/or type I collagen may be used and the polymerization step may be avoided. Because of the bonding strength of the adhesive of the present invention, only small amounts of the adhesive are required to seal a punctured artery. Moreover, because the surgical adhesive according to the present invention can polymerize almost immediately, the adhesive can polymerize on the surface and/or along the puncture track of the artery without penetrating the interior of the artery. Accordingly, large pieces or particles of material will not enter the circulatory system, thereby substantially reducing risk of embolism. Due to the fast and strong bonding of preferred adhesives of the invention, the patient will need to be immobilized for only a minimal period of time.

Formulations

The tissue treatment composition of the present invention may be presented in the same type of preparations as prior art fibrin sealants. The components may be provided in deep frozen solution form or as lyophilized powders, to be diluted prior to use with appropriate aqueous solutions, e.g. containing aprotinin and calcium ions, respectively. Additionally, the vascular sealants of the present invention may be formulated and shaped in the form of collagen plugs, as described in the art and known to one of ordinary skill in the art.

The compositions of the present invention can additionally comprise pharmaceutical agents, such as, for example, an antibiotic or a growth factor, by incorporating the agent into the tissue adhesive so as to be enclosed in the collagen network formed upon application of the tissue adhesive. The agent is thus kept at the site of application while being controllably released from the composition, such as when the composition is used as ocular drops, or a wound healing preparation, etc. As also mentioned above, the pharmaceutically active substance to be released from the present tissue adhesive composition may be the viscosity-enhancing polymer in itself or a substance coupled thereto. A specific example of such a viscosity-enhancing polymer fulfilling the viscosity enhancing requirement as well as having therapeutical and pharmaceutical utility, and in which it may be desired to sustain bioavailability, is hyaluronic acid and salts and derivatives thereof which are easily soluble in water and have an extremely short biological half-life. Thus, in one aspect, compositions of the present invention constitute an advantageous slow-release preparation for proteoglycans such as hyaluronic acid and its salts and derivatives, which considerably increases the bioavailability thereof.

Notably, the compositions of the present invention are not restricted to those having adhesive properties. Non-adhesive compositions are also included, especially when these compositions are primarily intended for wound healing. These compositions may in particular include non-adhesive proteins such as albumin and/or growth factors. Substantially non-adhesive compositions may also be obtained when the polymer part of the composition inhibits the adhesive properties of the protein part. It should in this context be emphasized that the invention comprises both adhesive and substantially non-adhesive compositions, although it has for simplicity reasons often has been referred to as an “adhesive” in this specification.

Application Of Compositions

The compositions of the present invention may be applied using a variety of dispensing devices. For example, the surgical adhesive may be applied using the devices set forth in U.S. Pat. Nos. 4,900,303 and 5,372,585, while monitoring the application process through an optical viewing system. The composition of the present invention may also be applied by the devices set forth in U.S. Pat. No. 5,129,882, or the other devices referenced above, or other devices as well known in the art.

Compositions according to the present invention may also be applied in conjunction with other sealing means. For example, adhesive compositions may be applied to puncture sites which have been closed using surgical sutures or tape, such as in the sealing of a puncture or incision in vascular tissues, including the heart. The adhesive in this instance will provide a complete seal, thereby reducing the risk of body fluid leakage from the organ or vessel, e.g., leakage from arterial puncture sites. The surgical adhesive of the present invention may additionally be used in conjunction with other sealing means, such as plugs, and the like. Such techniques are set forth in, for example, U.S. Pat. Nos. 4,852,568, 4,890,612, 5,053,046, 5,061,274, 5,108,421, 4,832,688, 5,192,300, 5,222,974, 5,275,616, 5,282,827, 5,292,332, 5,324,306, 5,370,660, and 5,021,059. The subject matter of these patents is incorporated herein by reference.

Notably, the compositions of this invention can be used to join together two surfaces by applying the particular composition to at least one of the surfaces. Depending on the particular requirements of the user, the adhesive compositions of this invention can be applied by known means, such as with, for example, a glass stirring rod, sterile brush, or medicine dropper, in many situations, a pressurized aerosol dispensing package is preferred in which the adhesive composition is in solution with a compatible anhydrous propellant. Aerosol application of the monomers is particularly advantageous for use in hemostasis. Mechanisms for aerosol applications are well known in the art.

### EXAMPLES

The following examples are provided solely to illustrate the claimed invention, and are not intended to limit the scope of the invention.

### Example 1

### Purification of Recombinant Type III Collagen from a Yeast Expression System

The following protocol was used to purify recombinant human type III collagen (rhc III or Rhc III) from Pichia pastoris:

1. Resuspend 1 volume of cell pellets with 7 volumes 0.1N HCL;

2. Fill Bead-Beater chamber half full with glass bead just taken from −20° C. freezer;

3. Fill the chamber with the cell suspension;

4. Assemble the chamber with an ice-water jacket;

5. Fill out the jacket with ice water with some sodium chloride;

6. Homogenize the cell pellets for 5×1 minutes, with 5 minute intervals between each 1 minute homogenization;

7. Recover the homogenate by filtration through a Buchner Funnel without filter paper;

8. Add pepsin solution to a final concentration of 0.2 mg/ml and incubate for 8 hours at 4° C.;

9. Centrifuge for 30 minutes at 10,000 rpm and collect the supernant (fraction S1) and pellet (fraction P1);

10. Adjust the pH to 7.4 with 10M NaOH, and incubate overnight at 4° C.;

11. Add 5M NaCl and HAC to 1M NaCl and 0.5M HAC;

12. Incubate at 4° C. for 1 hour;

13. Collect the pellets by centrifugation (fraction P2);

14. Dissolve the pellets in 3 volumes 0.1M HCl (depending on the collagen amount, adjust the collagen concentration to about 0.3 mg/ml);

15. Add 1.5 volumes of 3M urea, 0.3M NaCl, 0.15M Tris, pH 7.4, and adjust the pH to 7.4 with NaOH;

16. Run through DEAE-cellulose column (1.6×15 cm) at a flow rate of 0.1-0.2 ml/min;

17. Collect the flowthrough;

18. Concentrate the collagen by precipitation in 1M NaCl and 0.5M HAC;

19. Redissolve the pellet in 10mM HCl (fraction rhc III);

20. Dialyze the collagen solution against 10 mM HCl if necessary.

### Example 2

### Characterization of Recombinant Human Type III Collagen

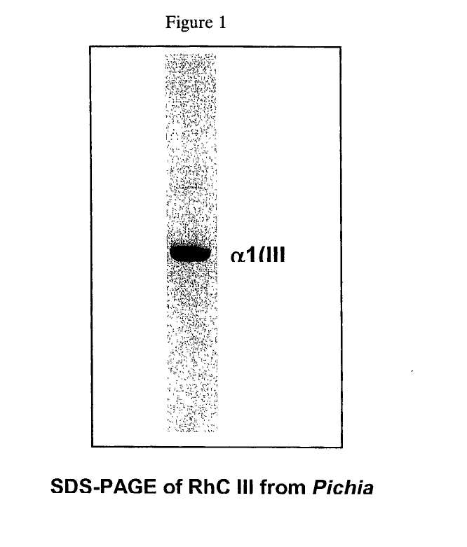

As defined above, purified rhc III was tested by SDS-PAGE as shown in FIG. 1. Amino acid analysis was performed and amino acid composition of purified rhc III is shown below in Table 1.

TABLE 1
Amino Acid Composition of Rhc III Purified from Pichia pastoris

| Amino acid | RhC III from Pichia pastoris | Human Type III Collagen |
|---|---|---|
| Asp | 46 | 42 |
| Glu | 68 | 71 |
| Hyp | 132 | 125 |
| Ser | 36 | 39 |
| Gly | 349 | 350 |
| His | — | 6 |
| Arg | 42 | 46 |
| Thr | 21 | 13 |
| Ala | 92 | 96 |
| Pro | 105 | 107 |
| Val | 14 | 14 |
| Met | 7 | 8 |
| Tyr | — | 3 |
| Ile | 14 | 13 |
| Leu | 20 | 22 |
| Hyl | 0 | 5 |
| Lys | 44 | 30 |
| Phe | 9 | 8 |
| Hyp/ | 0.557 | 0.538 |
| (Hyp + Pro) | | |

### Example 3

### Biocompatibility and Tissue Response Tests

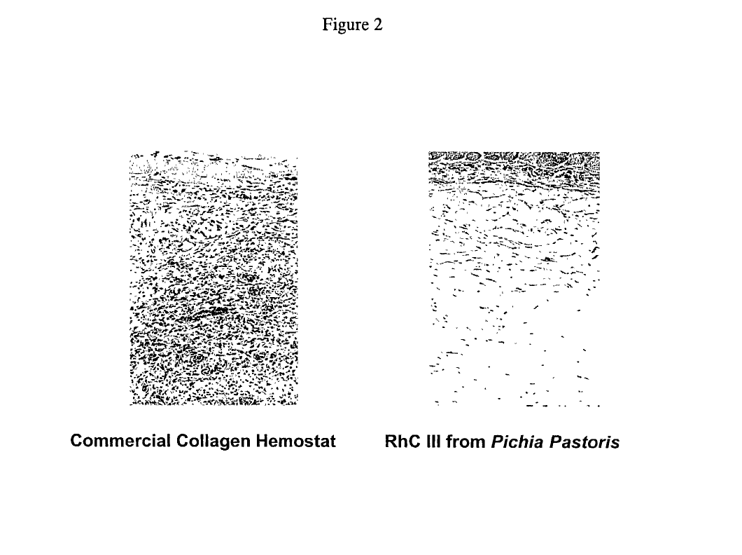

Biocompatibility and tissue response of rhc III and a commercially available collagen were tested in a rat subcutaneous model. Rhc III and a commercially available collagen hemostat were formulated into injectable paste/gel under sterile condition. The rhc III samples were tested to insure the endotoxin level is below the limit. The gel was injected subcutaneously into rats. The preliminary data indicates that Rhc III does not cause any erythema and edema. The gel was dissected at 2, 7, and 28 days following injection and examined histologically with H&E staining. The commercially available collagen hemostat has a much stronger tissue response than rhc III. A comparison of rhc III with the commercially available collagen hemostat harvested from rats on day 7 is shown in FIG. 2.

### Example 4

### Platelet Aggregation Test

The fibrillogenesis of above described recombinant human collagen type III (rch III) was tested according to the following method. Rhc III solution was diluted with 10 mM HCl to 1 mg/ml and 1/10 volume of 200 mM Na₂HPO₄, pH 11.2, was added. The solution was then mixed well and incubated at room temperature (20-22° C.) overnight. Following incubation, the fibril slurry was vortexed before usage.

Human Platelet Rich Plasma (PRP) was then prepared from fresh blood of an apparently healthy donor. The PRP was then adjusted to 200K/μl and the collagen fibril slurry was added into the PRP. Following addition of the fibril slurry, the platelet aggregation profile with an aggregometer was detected. The minimal amount of collagen to induce complete platelet aggregation is estimated from the amount of collagen to induce a fall in optical density of at least 30% occurred within 5 minutes.

The platelet aggregation capacity of recombinant human type III collagen was compared to the platelet aggregation capacity of bovine skin-derived collagen according to the methods set forth above and as more fully described in Balleisen, et al., 1975, Klin. Wschr 53:903-905, incorporated herein by reference. As set forth below in Table 2, fibrils generated from recombinant human type III collagen required a lower minimal amount to induce human platelet aggregation than that for fibrils generated from bovine skin collagen. Recombinant human type III collagen also has a shorter onset time to induce platelet aggregation. These results indicate that recombinant human collagen is a more hemostatic than tissue derived collagen.

TABLE 2

| | Minimal Amount for | |
|---|---|---|
| | Induction of Platelet | Time to Onset |
| Collagen Samples | Aggregation (μg) | (seconds) |
| Bovine Skin Collagen | 10 | 48 |
| Recombinant Human Type III | <5 | 36 |
| Collagen from Pichia | | |

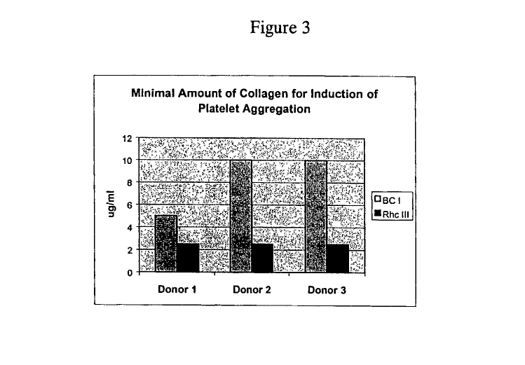

In a subsequent experiment, platelets were obtained from three healthy donors and were adjusted to 200K/ml in plasma. A collagen fibril slurry was added to the platelet suspension and the aggregation was measured with an Aggregometer. The minimal amount of collagen capable of inducing complete platelet aggregation was determined by stepwise decreasing the amount of collagen added. It was assumed that complete aggregation had occurred when a fall in OD of at least 30% occurred within 5 minutes. It was shown that rhc III has a lower minimal amount to induce complete aggregation of platelet, and indicates that rhc III is more hemostatic than bovine type I collagen. These results are depicted in FIG. 3 and demonstrate that type III collagen fibrils are more hemostatic than type I collagen fibrils.

### Example 5

### Hemostatic Effects of Rhc III Sponge

Rhc III was expressed in Pichia pastoris. A selected expression clone was cultivated in a bioreactor under defined conditions. Rhc III was purified from the harvested cell pellets by limited pepsin digestion and differential salt precipitation. Purified rhc III was formulated into fibrins at first by neutralization with phosphate buffer and incubation at room temperature overnight. The fibrils were collected by centrifugation and then resuspended in water. After homogenization, the rhc III gel was transferred into a mould and lyophilized into a sponge. This type of sponge has very poor water absorption.

Water absorptive capacity is critical for applications of rhc III as a hemostat and vascular sealant. To ensure the compositions of the present invention could satisfy this requirement, a process to formulate water absorptive rhc III sponge was developed. Essentially, a sponge was cross-linked, first with UV irradiation and then with 1% water-soluble carbodiimide. The residual cross-linking reagent was removed by incubation in PBS and the sponge was washed with water. Cross-linked sponges are lyophilized for animal testing and formulation of tissue sealant. This process not only enhanced the water absorption of rhc III sponge, but also significantly increased the mechanical strength of rhc III sponges. In a control study, bovine type I collagen was also formulated into sponges following the same procedures.

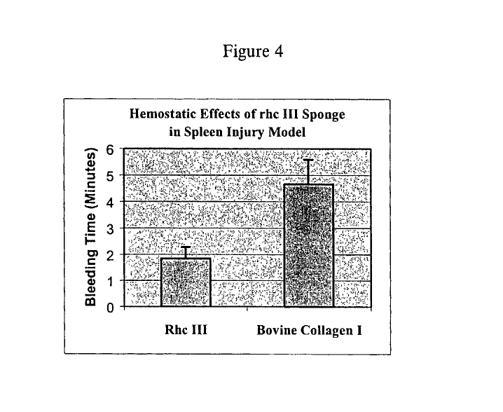

Acclimatized New Zealand White Rabbits were deeply anesthetized and laparotomies were performed to expose the spleens. Using a scalpel blade, uniform incisions about 1.0 cm long and 0.3 cm deep were made into the spleens. The incisions were then treated with rhc III sponges or with bovine type I collagen sponges. The time interval from the application of the test article or positive control material until the bleeding ceases was recorded. Statistical analysis of the mean time to hemostasis was performed using Anova and Student's two sample T-test. It was observed that the bleeding time of spleens treated with rhc III sponges was significantly shorter than that of those treated with bovine type I collagen. These results are shown in FIG. 4.

### Example 6

### Formulation of RhC III Sealant

To prepare a rhc III sealant, a three-step experimental approach was pursued: 1) preparing a cross-linked rhc III sponge; 2) coating the sponge with human thrombin; and 3) subsequently coating the sponge with human fibrinogen.

A collagen sponge cross-linked with water-soluble carbodiimide was rinsed with 100% ethanol and placed in a filtration funnel. Human thrombin suspended in ethanol (25 units/ml) was used to coat the sponge by filtration. The amount of thrombin on the sponge was about 10 units/cm². Human fibrinogen, dissolved in water at concentration of 4 mg/ml, was precipitated by mixing it with 3 volumes of ethanol. The precipitated fibrinogen was filtered through the sponge by vacuum. The amount of fibrinogen on the sponge was about 3 mg/cm². The coated sponge was lyophilized and used for animal tests.

Acclimatized New Zealand White Rabbits were deeply anesthetized and laparotomies were performed to expose kidneys and spleens. Using a scalpel blade, incisions about 1.0 cm long and 0.3 cm deep were made into the kidneys and/or spleens. The incisions were treated with commercially available INSTAT™ collagen sponge or rhc III sealant. The time interval from the application of the test article until the bleeding ceased was recorded. The adhesive capacity of testing articles was estimated by peeling the articles from the test site after hemostasis had been achieved. The results are shown below in Table 3 and Table 4.

TABLE 3
Hemostatic Effect and Adhesiveness of
Rhc III Sealant in Kidney Injury Model

| | Non-treatment | Instat ™ | Rhc III Sealant |
|---|---|---|---|
| Animal Numbers | 7 | 4 | 4 |
| Bleeding Time | 384 +/− 169 | 67 +/− 10 | <10 seconds* |
| (seconds) | | | |
| Adhesiveness** | ND | + | ++ |

*Bleeding stopped instantly upon application of the test articles
**+ weak, ++ medium, +++ strong.

TABLE 4
Hemostatic Effect and Adhesiveness of
Rhc III Sealant in Spleen Injury Model

| | Non-treatment | Instat ™ | Rhc III Sealant |
|---|---|---|---|
| Animal Numbers | 7 | 3 | 4 |
| Bleeding Time | 30.85 +/− 9.62 | 4.27 +/− 0.47 | <10 (seconds)* |
| | (minutes) | (minutes) | |
| Adhesiveness** | ND | + | ++ |

*Bleeding stopped instantly upon application of the test articles
**+ weak, ++ medium, +++ strong.

The results of these studies demonstrated that rhc III fibrils are able to induce human platelet aggregation at a lower concentration than bovine type I collagen, indicating that an rhc III sponge stops bleeding in spleen injury models within a shorter time period than bovine collagen sponges. A prototype of rhc III enhanced fibrin sealant showed a superior hemostatic potential in vivo over commercial collagen sponges.

### Example 7

### Cross-linking of Bovine Type I Collagen

A feasibility cross-linking test was conducted with soluble bovine type I collagen. Bovine type I collagen in 10 mM HCl was neutralized with 1/10 volume of 0.2M Na₂HPO₄, pH 11.2, and incubated overnight at room temperature. The fibrils were washed with water and concentrated to 50 mg/ml by centrifugation. The homogenized gel was transferred to a small cell culture dish and lyophilized to form a sponge.

To cross-link the collagen, the bovine collagen sponge was incubated in a solution of water-soluble carbodiimide at room temperature overnight, then washed with PBS and water. The cross-linked sponge was then dried and tested for solubility and water absorption, and compared to a commercially available collagen sponge (ANGIOSEAL™ collagen sponge). The data is shown in Table 5 below.

TABLE 5

| | | Solubility in | Water | |
|---|---|---|---|---|
| | | 10 mM HCl | Absorption | Wetting Time |
| Samples | Color | (% of control) | (mg/mg) | (seconds) |
| Angioseal ™ | White | 1.9% | 18.2 | 5 |
| Collagen | | | | |
| Sponge | | | | |

Water-soluble Carbodiimide Treated Vitrogen Sponge

| | | | | |
|---|---|---|---|---|
| 0% | White | 100% | 14.5 | >120 |
| 0.1% | White | 11.7% | 18.3 | >120 |
| 0.25% | White | 2.5% | 19.3 | 100 |
| 0.5% | White | 0% | 19.0 | 40 |
| 1% | White | 0% | 18.3 | 22 |
| 2% | White | 0% | 19.8 | 18 |

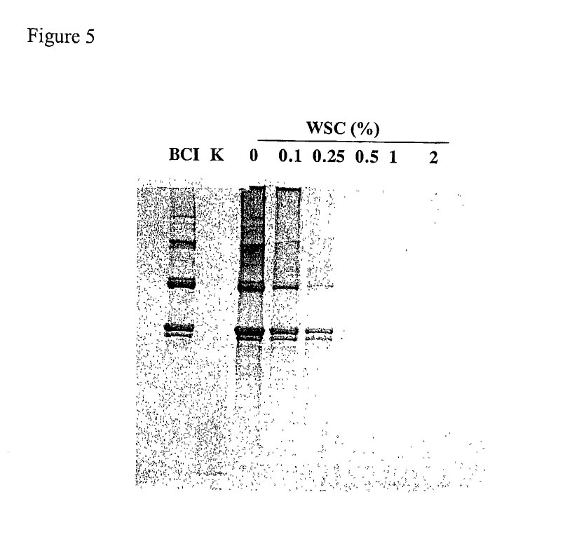

As shown in Table 5 and in FIG. 5, the cross-linked sponges appeared to be intact after incubation in water for 24 hours at room temperature, while the non-cross-linked collagen sponges were not intact. Using one's fingers, the cross-linked collagen sponges were split to test their mechanical strength. The collagen sponges cross-linked with water-soluble carbodiimide (WSC, as indicated in FIG. 5) at a concentration higher than 0.5% demonstrated reasonable mechanical strength.

### Example 8

### Cross-linking of Recombinant Type III Collagen

Rhc III collagen fibrils were prepared by neutralizing rhc III in 10 mM HCl with 1/10 volume of 0.2M Na₂HPO₄, pH 11.2, and incubated overnight at room temperature. Rhc III fibrils were harvested by centrifugation and washed with water. The fibrils were concentrated to 60 mg/ml and homogenized into a gel. The gel was transferred into a mold and lyophilized into a sponge with a thickness of about 2.5 mm. The collagen sponge was cross-linked with 1% ater-soluble carbodiimide in 25% ethanol at room temperature for 16 hours. Ethanol was added to accelerate the wetting of collagen sponges in the reaction solution. The cross-linked sponge was then washed with PBS for 2 hours, and subsequently washed three times with water. After lyophilization, collagen sponges of about 1.4 cm² in size were used to test for water reabsorption. In addition, 2 mg of the collagen sponge was used to test for solubility. As shown in Table 6, cross-linking of the rhc III sponge improved its water absorptive capacity. The wetting time of the cross-linked rhc III sponge was reduced to about 10 seconds, and water uptake increased from 76 mg to 433 mg. The cross-linked rhc III sponge remained intact after incubation in water for 24 hours at room temperature, while the non-cross-linked rhc III did not remain intact.

TABLE 6

| | | Solubility in | Water | Wetting |
|---|---|---|---|---|
| | | 10 mM HCl | Absorption | Time |
| Samples | Color | (% of control) | (mg in 90 seconds) | (seconds) |
| Angioseal ™ | White | 1.9% | 450 | 5 |
| Collagen | | | | |
| Sponge | | | | |
| Rhc III | White | 100% | 76 | >120 |
| Sponge | | | | |
| Cross-linked | White | 5.1% | 433 | 10 |
| Rhc III | | | | |
| Sponge | | | | |

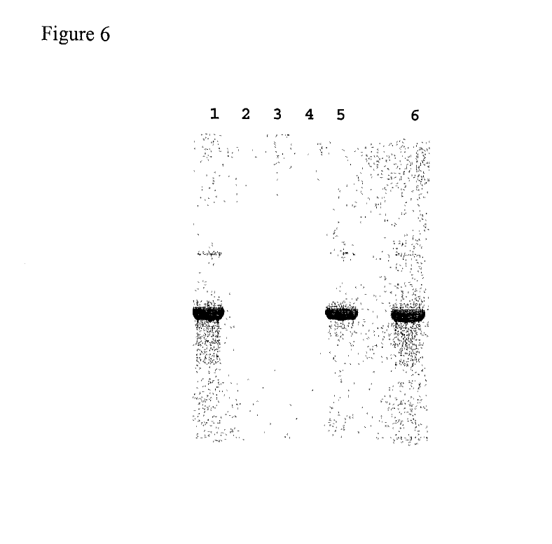

The collagen sponges were also tested with SDS-PAGE. As shown in FIG. 6, lane 1 and lane 6 represent rhc III; lane 2 represents the Angioseal™ collagen sponge; lane 3 represents cross-linked bovine type I collagen sponge; lane 4 represents cross-linked rhc III sponge; and lane 5 represents non-cross-linked rhc III sponge. The Angioseal™ collagen sponge, cross-linked bovine type I collagen sponge, and cross-linked rhc III sponge are not soluble in SDS-PAGE buffer.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims. All references cited herein are incorporated by reference herein in their entirety.

What is claimed is:

1. A composition useful as a hemostat, the composition comprising a collagen and an acceptable carrier, wherein the collagen consists essentially of type III collagen.

2. A composition useful as a hemostat, the composition comprising a collagen and an acceptable carrier, wherein the collagen consists essentially of human type III collagen.

3. A composition useful as a hemostat, the composition comprising a collagen and an acceptable carrier, wherein the collagen consists essentially of recombinant type III collagen.

4. A composition useful as a hemostat, the composition comprising a collagen and an acceptable carrier, wherein the collagen consists essentially of recombinant human type III collagen.

5. The composition of claim 1, wherein the type III collagen consists essentially of type III collagen monomers.

6. The composition of claim 1, wherein the type III collagen consists essentially of type III collagen polymers.

7. A hemostat comprising the composition of claim 1.

8. A sealant comprising the composition of claim 1.

9. The sealant of claim 8, wherein the sealant is selected from the group consisting of a vascular sealant or a tissue sealant.

10. A wound dressing comprising the composition of claim 1.

11. A sponge comprising the composition of claim 1.

12. An adhesive comprising the composition of claim 1.

13. The adhesive of claim 12, wherein the adhesive is selected from the group consisting of a tissue adhesive, a surgical adhesive, or a vascular adhesive.

14. A method of achieving hemostasis in a subject having a wound, the method comprising administering at the site of the wound the composition of claim 1 under conditions suitable for achieving hemostasis.

15. A method of reducing bleeding in a subject having a wound, the method comprising administering at the site of the wound the composition of claim 1 under conditions suitable for reducing bleeding.

16. A method of sealing a wound in a subject having a wound, the method comprising administering at the site of the wound in need of sealing the composition of claim 7 under conditions suitable for sealing the wound.

17. A method of sealing a puncture in a subject having a puncture, the method comprising administering at the site of the puncture in need of sealing the composition of claim 7 under conditions suitable for sealing the puncture.

18. A method of sealing an incision in a subject having an incision, the method comprising administering at the site of the incision in need of sealing the composition of claim 7 under conditions suitable for sealing the incision.

19. A method for preparing a composition useful as a hemostat, the method comprising:
(a) providing collagen, wherein the collagen consists essentially of type III collagen;
(b) providing an acceptable carrier; and
(c) mixing the collagen and the carrier to form the composition.

20. The method of claim 19, wherein the type III collagen is a recombinant type III collagen.

21. The method of claim 20, wherein the recombinant type III collagen is a recombinant human type III collagen.
